(19) United States
(12) Patent Application Publication
(10) Pub. No.: US 2024/0000987 A1
(43) Pub. Date: Jan. 4, 2024

(54) ULTRAVIOLET LIGHT BASED STERILIZING CABINET

(71) Applicant: WEISMACHER ECO PRIVATE LIMITED, Ahmedabad (IN)

(72) Inventors: Rajeshkumar Hariprasad Acharya, Ahmedabad (IN); Yusuf Nazmuddin Ghadiali, Ahmedabad (IN)

(73) Assignee: WEISMACHER ECO PRIVATE LIMITED, Ahmedabad (IN)

(21) Appl. No.: 18/254,443

(22) PCT Filed: Nov. 20, 2021
(86) PCT No.: PCT/IN2021/051087

§ 371 Date: May 25, 2023

(30) Foreign Application Priority Data
Nov. 26, 2020 (IN) 202021051480

(51) Int. Cl. A61L 2/10; A61L 2/26

(52) U.S. Cl. CPC A61L 2/10; A61L 2202/122; A61L 2/26

(57) ABSTRACT

A cabinet for sterilizing a belongings according to the present invention comprises; the cabinet body having a door in the front; A layer of silver foil is applied on the all side of inner walls of the cabinet including door; An ultra-violet lights are placed on the inner wall of the door and all sides of the inner walls of the cabinet; A glass interior being fixed after placing the ultra-violet lights inside the cabinet including door; the ultra-violet sterilization lights emits ultraviolet rays to the sterilization chamber; Sterilization means for sterilizing a sterilization target object placed on the glass interior of the cabinet; A magnetic switch is mounted at the top of inner wall of the door and opposite wall of the cabinet and an indicator mounted on the door.

### FIELD OF INVENTION

The present invention relates to a cabinet for sterilizing the belongings, and more specifically, sterilizing various items and belongings (hereinafter, referred to as sterilization objects) such as bags, books, toys, dolls, mobile phones, wallets, cards, bills, coins, shoe insoles, feeding bottles, and the like. The invention relates to a cabinet for sterilizing the belongings, which is most suitable for use and has high space utilization.

### BACKGROUND OF INVENTION

Generally, personal belongings such as writing instruments or mobile phones, small loan items such as toys or dolls, and various currencies such as banknotes or coins may also be used as various pathogenic bacteria and molds due to contact of the above items by exchange and exchange between users and other users. This can cause disease.

As described above, bacteria causing disease are propagated in belongings such as writing instruments, mobile phones, toys, and bills that can be carried by individuals.

Conventionally, a book sterilizer configured to sterilize with an ultraviolet lamp or to mount a book on a book and discharge air with a blower is available on the market.

However, recently, among the general public, the awareness of bacteria has increased, and more people are required to sterilize various portable goods besides books.

In addition, it is difficult to purchase an expensive sterilizer for sterilizing personal portable goods, and most sterilizers are difficult to use at home because they are large in size.

For this, the present invention provides a cabinet for sterilizing the belongings by irradiating ultraviolet light to these personal belongings, since a very large number of bacteria can be eliminated by this radiation. The integration of ultraviolet radiation in conventional supports for general goods, the present invention allows obtaining a new final product that meets the necessary requirements to ensure correct asepsis of personal belongings.

### OBJECT OF INVENTION

The main object of the present invention is to provide a cabinet for sterilizing the belongings and to provide a mounting means corresponding to each sterilization object so that the sterilization object can be mounted in the sterilization cabinet to easily sterilize the sterilization object.

Another object of the present invention is to provide minimized space to make the overall size of the product compact, so that it can be miniaturized to be most suitable for general use.

Still another object of the present invention is to providing a versatile sterilizer with high space utilization.

### SUMMARY OF INVENTION

In order to achieve the above object, the present invention is related to a cabinet for sterilizing the belongings wherein sterilization chamber is formed therein the cabinet body having a door in the front; A layer of silver foil is applied on the all side of inner walls of the cabinet including door; An ultra-violet lights are placed on the inner wall of the door and all sides of the inner walls of the cabinet; A glass interior being fixed after placing the ultra-violet lights inside the cabinet including door; the ultra-violet sterilization lights emits ultraviolet rays to the sterilization chamber; Sterilization means for sterilizing a sterilization target object placed on the glass interior of the cabinet; A magnetic switch is mounted at the top of inner wall of the door and opposite wall of the cabinet and an indicator mounted on the door.

There is an advantage that can easily sterilize various sterilization objects, and also can minimize the space of the sterilization room to compact the overall size of the product can be easily used at offices, hospitals, schools, colleges, restaurants, hotels, internet coffee shops, malls etc. As a result, the price of the product is low and there is an advantage that the general public can feel free to purchase.

### BRIEF DESCRIPTION OF DRAWINGS

FIG. 1 illustrates a front view of a cabinet for sterilizing the belongings according to present invention.

FIG. 2 illustrates an inside view of a cabinet for sterilizing the belongings according to present invention.

### DETAIL DESCRIPTION OF INVENTION

Before explaining the present invention in detail, it is to be understood that the invention is not limited in its application to the details of the construction and arrangement of parts illustrated in the accompany drawings. The invention is capable of other embodiment, as depicted in different figures as described above and of being practiced or carried out in a variety of ways. It is to be understood that the phraseology and terminology employed herein is for the purpose of description and not of limitation.

It is to be also understood that the term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also contain one or more other components.

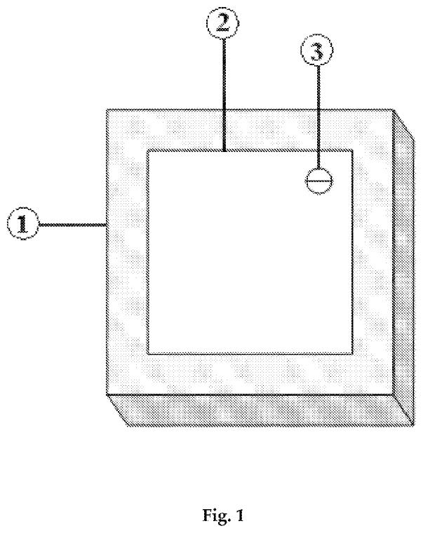

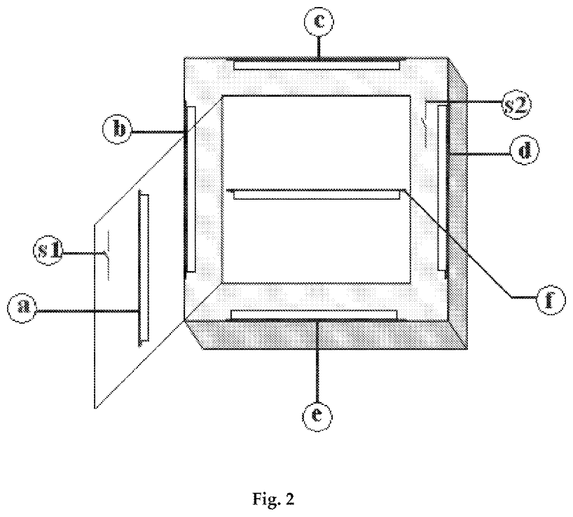

FIGS. 1 and 2 illustrate the front and inside view of the cabinet for sterilizing the belongings according to present invention. As shown in said FIG. 1, the cabinet according to present invention mainly comprises a cabinet body (1) having a door (2); an indicator (3) being placed top on the door for visualize the process of the sterilizing of the belongings.

As shown in FIG. 2, the fig. shows the inside view of the cabinet according to present invention. A cabinet body (1) having six inner walls inside the cabinet including door (2). Each inner wall of the cabinet body (1) having a layer of metal foil (not shown). A ultra-violet lights (a, b, c, d, e, f) are placed in each side of the inside walls of the cabinet body (1). A glass interior (not shown) fixed at each side of inner walls of the cabinet. A magnet switch (s1, s2) placed at the closing end of inner wall of the door (2) and the opposite wall of the cabinet body (1). An electric box (not shown) fixed at the back side of cabinet body (1) to provide electric power supply.

In working of the cabinet for sterilizing the belongings according to present invention, the door is opened and the belongings to be sterilized are placed into the chamber. A sterilization target object placed inside the glass interior of the cabinet. By closing the door (2), the magnetic switches (s1, s2) make the close contact and generate the electric signals and turn on the ultra-violet lights (a, b, c, d, e, f). The ultra violates sources produces and radiates UV rays on the object that disinfect or sanitize infestation agents within various cleaning media by ultraviolet radiation.

The invention has been explained in relation to specific embodiment. It is inferred that the foregoing description is only illustrative of the present invention and it is not intended that the invention be limited or restrictive thereto. Many other specific embodiments of the present invention will be apparent to one skilled in the art from the foregoing disclosure.

All substitution, alterations and modification of the present invention which come within the scope of the following claims are to which the present invention is readily susceptible without departing the invention. The scope of the invention should therefore be determined not with reference to the above description but should be determined with reference to appended claims along with full scope of equivalents to which such claims are entitled.

### LIST OF REFERENCE NUMERALS

1 Cabinet Body
2 Door
3 Indicator
S a Ultra-Violet light
b Ultra-Violet light
c Ultra-Violet light
d Ultra-Violet light
e Ultra-Violet light
f Ultra-Violet light
s1 Magnetic Switch
s2 Magnetic Switch

1. An ultraviolet light based sterilizing cabinet for sterilizing the belongings comprises a cabinet body (1) having a door (2) and an indicator (3);
said cabinet body (1) having six inner walls inside the cabinet including door (2);
characterized in that,
wherein each inner wall of the cabinet body (1) having a layer of metal foil and a glass interior fixed at each side of inner walls of the cabinet;
wherein a ultra-violet lights (a, b, c, d, e, f) are placed in each side of the inside walls of the cabinet body (1);
wherein a magnet switches (s1, s2) is placed at the closing end of inner wall of the door (2) and the opposite wall of the cabinet body (1);
wherein an electric box fixed at the back side of cabinet body (1) to provide electric power supply.

2. A method for sterilizing the belongings from ultraviolet light based sterilizing cabinet as claimed in claim 1 comprising following steps;
a. Opening the door and placing the belongings into the chamber of the glass interior of the cabinet for sterilizing;
b. closing the door (2) and the making the close contact of the magnetic switches (s1, s2) for generating the electric signals to turn on the ultra-violet lights (a, b, c, d, e, f);
c. producing the ultra violates sources and radiating UV rays on the object that disinfect or sanitize infestation agents within various cleaning media by ultraviolet radiation.
